(19) United States
(12) Patent Application Publication
(10) Pub. No.: US 2020/0390391 A1
(43) Pub. Date: Dec. 17, 2020

(54) FALSE TOOTH DEVICE, OPERATING METHOD THEREOF, TERMINAL, AND SIGNAL INTERACTION SYSTEM

(71) Applicant: BOE TECHNOLOGY GROUP CO., LTD., Beijing (CN)

(72) Inventor: Wenbo LI, Beijing (CN)

(73) Assignee: BOE Technology Group Co., Ltd., Beijing District (CN)

(21) Appl. No.: 16/070,411

(22) PCT Filed: Jan. 11, 2018
(86) PCT No.: PCT/CN2018/072273

§ 371 Date: Jul. 16, 2018

(30) Foreign Application Priority Data
Jun. 2, 2017 (CN) 201710409756.5

(51) Int. Cl. A61B 5/00; A61B 5/04

(52) U.S. Cl. CPC A61B 5/682; A61B 5/0015; A61B 5/0004; A61B 5/04; A61B 2560/04; A61B 2562/0219; A61B 2562/0247; A61B 2560/0214; A61B 5/749

(57) ABSTRACT

The disclosure discloses a false tooth device, an operating method thereof, a terminal, and a signal interaction system so as to enable the false tooth device to achieve functions of detecting and processing a signal. A false tooth device according to the disclosure includes a false tooth body, and a processing component, an interaction component, and a detection component, which are arranged in the false tooth body, wherein the detection component is configured to transmit a detected signal to the processing component; the processing component is configured to identify the received signal as a signal capable of interacting with the interaction component, and to transmit the signal to the interaction component; and the interaction component is configured to output the signal transmitted by the processing component.

This application is a National Stage of International Application No. PCT/CN2018/072273, filed Jan. 11, 2018, which claims priority to Chinese Patent Application No. 201710409756.5, filed Jun. 2, 2017, both of which are hereby incorporated by reference in their entireties.

### FIELD

The present disclosure relates to the field of wearable devices, and particularly to a false tooth device, an operating method thereof, a terminal, and a signal interaction system.

### BACKGROUND

With the advancement of the society and the living standard, people are paying more attention to their living qualities and physical health conditions. However, it may be difficult for the people to identify hidden physical discomforts if there are no physical symptoms, and these discomforts may tend to be ignored particularly by office workers with a quick rhythm of life and a significant stress due to their working, thus possibly resulting in a hidden risk of physical health. Accordingly a solution to detecting a physical health signal anytime is highly desirable. The mouths of people are working all the time, e.g., coughing, chewing, swallowing, gnashing, speaking, etc., where all of these spontaneous movements may indicate a physical health signal, or that people are going to interact with a terminal via a signal; and the teeth which are the closest to the mouths can perceive their movements the best.

A false tooth is a tooth restoration, and since the existing false tooth only has a function of aiding its wearer in chewing, its function is very single.

### SUMMARY

Embodiments of the disclosure provide a false tooth device including a false tooth body, and a processing component, an interaction component, and a detection component, which are arranged in the false tooth body, wherein: the detection component is connected with the processing component, and configured to transmit a detected signal to the processing component; the processing component is connected with the interaction component, and configured to identify a received signal as a signal capable of interacting with the interaction component, and to transmit the received signal to the interaction component; and the interaction component is configured to output a signal transmitted by the processing component.

Optionally the interaction component includes a radio communication component; the processing component is configured to identify the received signal as a signal capable of interacting with the radio communication component, and to transmit the received signal to the radio communication component; and the radio communication component is configured to output the received signal for interaction to a corresponding terminal.

Optionally the radio communication component includes at least one of following components: a Bluetooth component, a Wireless-Fidelity (Wi-Fi) component, a Zigbee component, a General Packet Radio Service (GPRS) component, and a radio frequency component.

Optionally the interaction component includes a broadcasting component; the processing component is configured to identify a signal transmitted by the detection component as a voice signal, and to transmit the voice signal to the broadcasting component; and the broadcasting component is configured to output the voice signal.

Optionally the broadcasting component includes a speaker; the processing component is configured to identify the signal transmitted by the detection component as the voice signal, and to transmit the voice signal to the speaker; and the speaker is configured to make a voice corresponding to the voice signal.

Optionally the false tooth device further includes a storage component configured to store a correspondence relationship between the signal detected by the detection component, and the signal capable of interacting with the radio communication component; and the processing component is configured to identify the signal detected by the detection component as the signal capable of interacting with the radio communication component according to the correspondence relationship.

Optionally the false tooth device further includes a power component configured to provide power.

Optionally the detection component includes at least one of following sensors: an acceleration sensor, a gyroscope sensor, a pressure sensor, and a Global Positioning System (GPS) sensor.

Optionally the interaction component further includes a speaker; the radio communication component is further configured to receive a control signal transmitted by the terminal, and to transmit the control signal to the processing component; the processing component is further configured to identify the control signal as a voice signal, and to transmit the voice signal to the speaker; and the speaker is configured to make a voice corresponding to the voice signal.

Embodiments of the disclosure provide a terminal including a communication element configured to interact with the false tooth device, and a processing element configured to process a signal received from the false tooth device, and/or to transmit a signal to the false tooth device through the communication element.

Optionally the terminal includes at least one of following terminals: a TV set, a mobile phone, a tablet computer, a watch, and a brain wave detection device.

With the terminal according to the embodiments of the disclosure, a user can interact with the TV set, the mobile phone, or another external terminal via a signal through the false tooth device, and can manipulate the TV set, the mobile phone, or another device through hitting the false tooth or otherwise instead of contacting with the TV set, the mobile phone, or another device.

Embodiments of the disclosure provide a signal interaction system including the false tooth device according to any one of the embodiments above of the disclosure, and the terminal according to the embodiments above of the disclosure.

Optionally when the interaction component includes a radio communication component and a speaker, and the terminal includes a brain wave detection device, the brain wave detection device is configured to transmit an acquired brain wave signal to the processing component in the false tooth device through the radio communication component; the processing component is configured to identify the brain wave signal as a voice signal, and to transmit the voice signal to the speaker; and the speaker is configured to make a voice corresponding to the voice signal.

Embodiments of the disclosure provide an operating method of the false tooth device, the method including: transmitting, by the detection component, the detected signal to the processing component; identifying, by the processing component, the signal detected by the detection component as the signal capable of interacting with the interaction component, and transmitting the signal to the interaction component; and outputting, by the interaction component, the signal transmitted by the processing component.

### BRIEF DESCRIPTION OF THE DRAWINGS

FIG. 1 is a schematic structural diagram of a false tooth device according to embodiments of the disclosure;

FIG. 2 is a first schematic circuit diagram of the false tooth device according to the embodiments of the disclosure;

FIG. 3 is a second schematic circuit diagram of the false tooth device according to the embodiments of the disclosure;

FIG. 4 is a third schematic circuit diagram of the false tooth device according to the embodiments of the disclosure;

FIG. 5 is a schematic circuit diagram of a terminal according to the embodiments of the disclosure; and

FIG. 6 is a schematic circuit diagram of a device for detecting a brain wave according to the embodiments of the disclosure.

### DETAILED DESCRIPTION OF THE EMBODIMENTS

Embodiments of the disclosure provide a false tooth device so as to realize a false tooth device which can transmit a corresponding signal through movements of a mouth, and interact with a terminal via the signal, and to enable the terminal interacting with the false tooth device via the signal to convey in effect a sound through the false tooth device to thereby facilitate a person vocally disabled.

The embodiments of the disclosure provide a false tooth device including a false tooth body, and a processing component, an interaction component, and a detection component, which are arranged in the false tooth body, where the processing component can be a central processing unit, an application-specific integrated circuit, a complex programmable logic device, or another processor which can process a signal; the interaction component can include a radio communication component and/or a speaker (a broadcasting component), and can further include another component or other components; and the detection component can be a sensing device, or can be another device which can detect a signal. Particularly the detection component transmits a detected signal to the processing component, and the processing component identifies the signal as a signal capable of interacting with the interaction component, and transmit the signal to the interaction component.

The false tooth device according to the embodiments of the disclosure will be described below with reference to the drawings.

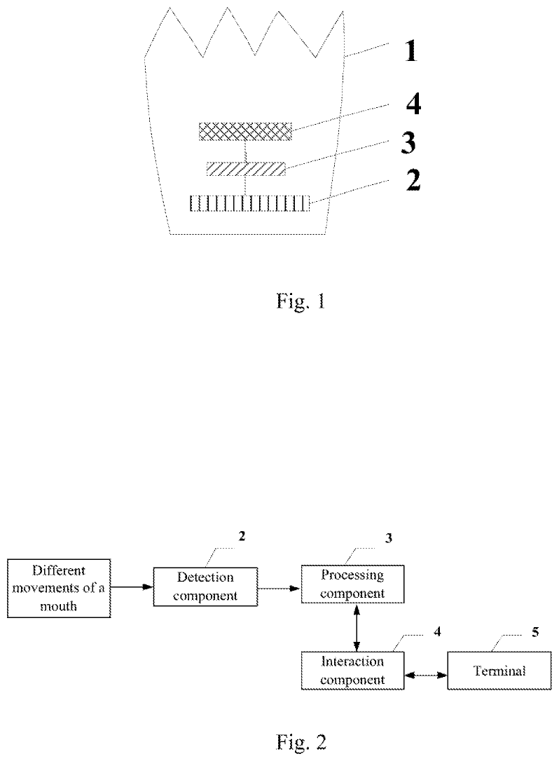

As illustrated in FIG. 1, a false tooth device according to the embodiments of the disclosure includes a false tooth body 1, and a detection component 2, a processing component 3, and an interaction component 4, which are arranged in the false tooth body 1.

The detection component 2 is connected with the processing component 3, and configured to transmit a detected signal to the processing component 3.

The processing component 3 is connected with the interaction component 4, and configured to identify the received signal as a signal capable of interacting with the interaction component 4, and to transmit the received signal to the interaction component 4.

The interaction component 4 is configured to output a signal transmitted by the processing component 3.

Particularly in the false tooth device above according to the embodiments of the disclosure, since the processing component 3, the interaction component 4, and the detection component 2 are arranged in the false tooth body 1, there may be additional functions of the false tooth device so that the false tooth device can be provided with more functions, and can detect a signal and process a signal.

Particularly as illustrated in FIG. 2, an operating principle of the false tooth device above according to the embodiments of the disclosure is that the detection component 2 transmits a detected signal to the processing component 3 when the mouth moves; the processing component 3 identifies the received signal as a signal capable of interacting with the interaction component 4, and transmits the received signal to the interaction component 4; and the interaction component 4 outputs the signal transmitted by the processing component 3 to a terminal 5.

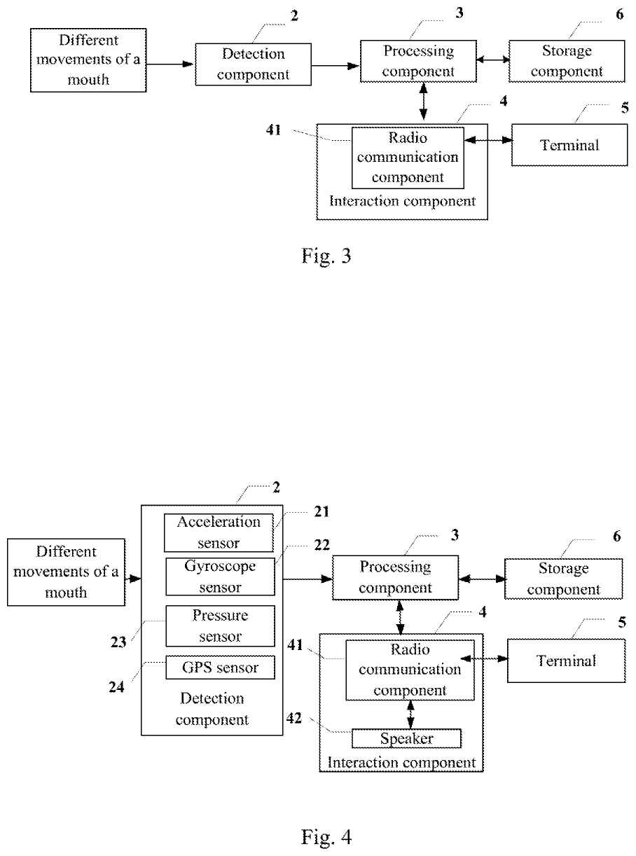

Optionally in the false tooth device above according to the embodiments of the disclosure, as illustrated in FIG. 3, the interaction component 4 includes a radio communication component 41.

The processing component 3 is configured to identify the received signal as a signal capable of interacting with the radio communication component 41, and to transmit the received signal to the radio communication component 41.

The radio communication component 41 is configured to output the interactive signal to a corresponding terminal 5, and to interact with the terminal 5 via the signal.

Optionally in the false tooth device above according to the embodiments of the disclosure, the radio communication component 41 includes one or a combination of the following components: a Bluetooth component, a Wireless-Fidelity (Wi-Fi) component, a Zigbee component, a General Packet Radio Service (GPRS) component, a radio frequency component, or another component for wireless communication, although a repeated description thereof will be omitted here.

Optionally in the false tooth device above according to the embodiments of the disclosure, the interaction component 4 further includes a broadcasting component.

The processing component 3 is configured to identify the signal transmitted by the detection component 2 as a voice signal, and to transmit the voice signal to the broadcasting component; and the broadcasting component is configured to output the voice signal.

Optionally in the false tooth device above according to the embodiments of the disclosure, as illustrated in FIG. 4, the broadcasting component includes a speaker 42.

The processing component 3 is configured to identify the signal transmitted by the detection component 2 as a voice signal, and to transmit the voice signal to the speaker 42; and the speaker 42 is configured to make a voice corresponding to the voice signal.

Particularly in the false tooth device above according to the embodiments of the disclosure, the detection component 2 transmits the detected signal to the processing component 3, and the processing component 3 identifies the signal as a signal capable of interacting with the interaction component 4, and then interacts with the outside through the radio communication component 41. When the signal is identified as a health condition signal, the processing component 3 transmits health parameter information of a user to the terminal 5 for display through the radio communication component 41, so that the user can monitor his or her own physical health condition conveniently any time, e.g., an appetite condition, an emotional condition, falling sick or not, etc. When the signal is identified as a control signal of the user on the terminal 3, the processing component 3 performs a corresponding operation on the terminal 5 through the radio communication component 41, for example, to control the volume of a TV set to be raised or lowered, to tune a program channel, to move an unpiloted aircraft to move upward or downward, etc. When the signal is identified as a voice signal, a corresponding voice is made through the speaker 42 to aid the user in speaking so as to facilitate a person who is vocally disabled.

Optionally in the false tooth device above according to the embodiments of the disclosure, as illustrated in FIG. 3, the false tooth device further includes a storage component 6 configured to store a correspondence relationship between a signal detected by the detection component 2, and a signal capable of interacting with the radio communication component 41.

The processing component 3 is configured to identify the signal detected by the detection component 2 as the signal capable of interacting with the radio communication component 41 according to the correspondence relationship, to output the signal through the radio communication component 41, for example, to the terminal 5, and to interact with the terminal 5 via the signal.

Particularly different control signal instructions can be preset in the storage component 6, and for example, hitting the false tooth body 1 once indicates that the terminal 5 is to be enabled, hitting the false tooth body 1 twice indicates that the terminal 5 is to be disabled, etc. The detection component 2 detecting the movement of the false tooth body 1 outputs a signal to the processing component 3; the processing component 3 conveys a preset instruction signal, corresponding to the signal detected by the detection component 2, stored in the storage component 6 to the radio communication component 41; and thereafter the radio communication component 41 outputs the instruction signal to the terminal 5, and interacts with the terminal 5 via the signal to thereby perform an operation on the terminal 5.

Optionally in the false tooth device above according to the embodiments of the disclosure, the false tooth device further includes a power component configured to provide power.

Optionally in the false tooth device above according to the embodiments of the disclosure, the power component includes an accumulator.

Optionally in the false tooth device above according to the embodiments of the disclosure, the accumulator is charged wirelessly.

At present, the technology of a chip embedded in a human body has been applied for years, and particularly the chip is embedded into the skin, the brain, and even an organ of the human body to detect a physical health condition. At present, as for charging in a human body, Stanford University, U.S.A., has successfully powered wirelessly a micro powered element embedded in the human body at a depth of approximately 5 cm below the skin of the human body at a high frequency of 1.7 GHz, where power of 50 μW is successfully transmitted to the powered element with a diameter of approximately 0.8 mm at the depth of 5 cm in the human body through energy transmission of electromagnetic induction and an electromagnetic wave in combination to power wirelessly a heart pacemaker, a swallowed camera in a digestive system, a manmade inner ear, a wireless tube embedded in a blood vessel, or other micro medical elements.

Hereupon the false tooth device above according to the embodiments of the disclosure can be a removable false tooth, or can be a fixed false tooth. For the sake of safety, when it is a fixed false tooth, it is preferably charged wirelessly, although the embodiments of the disclosure will not be limited thereto; and when it is a removable false tooth, it can be charged in a wired or wireless manner outside the human body, although the embodiments of the disclosure will not be limited thereto.

It shall be noted that the false tooth device 1 according to the embodiments of the disclosure can further include other structural components, and the interaction component 4 can further include other components, although a repeated description thereof will be omitted here.

Optionally in the false tooth device above according to the embodiments of the disclosure, as illustrated in FIG. 4, the detection component 2 includes one or a combination of the following sensors: an acceleration sensor 21, a gyroscope sensor 22, a pressure sensor 23, and a Global Positioning System (GPS) sensor 24.

Particularly the acceleration sensor 21 can cooperate with the gyroscope sensor 22 to detect the frequency of movements of the mouth, e.g., coughing, chewing, swallowing, gnashing, speaking, etc., to thereby conclude a physical health condition. For example, a statistic is made to calculate the frequency of coughing to thereby conclude whether the user has caught a cold; a statistic is made to calculate the number of times, and the frequency of chewing to thereby conclude an appetite condition of the user; and an emotional condition of the user is determined according to the frequency of speaking.

The pressure sensor 23 is configured to conclude the extent of stress of the user, and can be further configured to convey a signal related to hitting between upper and lower teeth of the user, and for example, the strength or frequency of hitting can represent a different control signal which can be used to control the terminal 5.

The GPS sensor 24 is configured to position the user, and for example, an elder, or a memory disabled person can be positioned to prevent him or her from getting lost.

Optionally in the false tooth device above according to the embodiments of the disclosure, as illustrated in FIG. 4, the interaction component 4 further includes a speaker 42.

The radio communication component 41 is further configured to receive a control signal transmitted by the terminal 5, and to transmit the control signal to the processing component 3.

The processing component 3 is further configured to identify the control signal as a voice signal, and to transmit the voice signal to the speaker 42.

The speaker 42 is configured to make a voice corresponding to the voice signal.

Particularly in the false tooth device above according to the embodiments of the disclosure, the radio communication component 41 receives a control signal transmitted from the outside, e.g., a control signal transmitted by the terminal 5, and transmits the control signal to the processing component 3 in the false tooth device, and the processing component 3 identifies the control signal as a voice signal, and makes a corresponding voice through the speaker 42 to thereby facilitate a person vocally disabled. Particularly the terminal 5 can be one or a combination of a TV set, a mobile phone, a tablet computer, a watch, and a brain wave detection device.

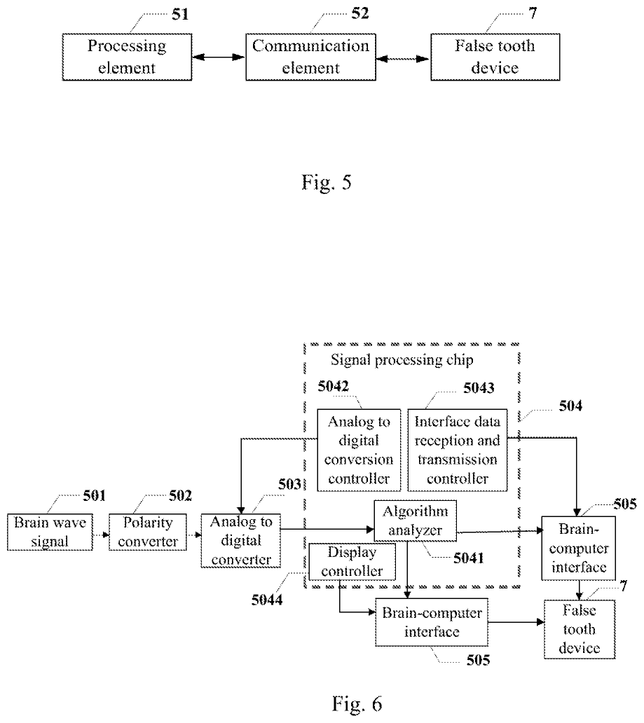

Based upon the same inventive concept, the embodiments of the disclosure further provides a terminal as illustrated in FIG. 5, which includes a communication element 52 configured to perform signal interaction with a false tooth device 7, and a processing element 51 configured to process a signal received from the false tooth device 7, and/or to transmit a signal to the false tooth device 7 through the communication element 52, where the false tooth device 7 is the false tooth device according to any one of the embodiments above of the disclosure.

Here the communication element 52 can be a Bluetooth component, a Wi-Fi component, a Zigbee component, a GPRS component, a radio frequency component, or another component for radio communication; and the processing element 52 can be a central processing unit, an application-specific integrated circuit, a complex programmable logic device, or another processor which can process a signal.

Optionally in the terminal above according to the embodiments of the disclosure, the terminal includes one or a combination of the following terminals: a TV set, a mobile phone, a tablet computer, a watch, and a brain wave detection device.

Particularly the user can interact with the TV set, the mobile phone, or another external terminal via a signal through the false tooth device 7, and can manipulate the TV set, the mobile phone, or another device through hitting the false tooth or otherwise instead of contacting with the TV set, the mobile phone, or another device.

Based upon the same inventive concept, the embodiments of the disclosure further provide a signal interaction system including the false tooth device according to any one of the embodiments above of the disclosure, and the terminal capable of interacting with the false tooth device via a signal according to any one of the embodiments above of the disclosure.

Optionally in the signal interaction system above according to the embodiments of the disclosure, when the interaction component includes the radio communication component and the speaker, and the terminal includes the brain wave detection device, the brain wave detection device is configured to transmit an acquired brain wave signal to the processing component in the false tooth device through the radio communication component; the processing component is configured to identify the brain wave signal as a voice signal, and to transmit the voice signal to the speaker; and the speaker is configured to make a voice corresponding to the voice signal.

Particularly in the signal interaction system above according to the embodiments of the disclosure, the brain wave detection device and the false tooth device cooperate with each other so that a user can operate on a terminal through the false tooth device, can further convey an idea of the user through the terminal, and can convey in effect a sound through the false tooth device to thereby facilitate a person vocally disabled.

A particular operating process of the brain wave detection device will be described below as follows.

As illustrated in FIG. 6, the working brain produces a brain wave signal 501, a potential difference is created between cortex cell groups of the brain to thereby produce current outside the cortex cells of the brain; a polarity converter 502 can obtain a corresponding brain wave analog signal through an embedded or external electrode; an analog to digital converter 503 converts the analog signal into a digital signal; an algorithm analyzer 5041 on a signal processing chip 504 algorithmically analyzes the digital signal, and then presents the brain wave signal 501 at different band frequencies, where the brain wave signal 501 at the different band frequencies correspond to different conditions of a person, or express his or her different ideas; and a brain-computer interface 505 analyzes the signal at the respective band frequencies, and obtains corresponding data information or instruction signals, and then interacts with the false tooth device via the signals; where the signal processing chip 504 further includes an analog to digital conversion controller 5042, an interface data reception and transmission controller 5043, a display controller 5044, etc.; and there may be a plurality of brain-computer interfaces 505. The brain wave detection device is well known in the prior art, so a repeated description thereof will be omitted here.

Particularly the brain wave detection device determine the condition of the user, e.g., stressed, relaxed, etc., and can further determine a specific stressed part of the brain through a plurality of electrodes, e.g., the front part, the back part, the left part, or the right part of the brain, etc. Different voice instructions can be preset for the different parts of the brain, and for example, if the front part of the brain is working, then it will indicate that “I want to drink”; if the back part of the brain is working, then it will indicate that “I am hungry and want to eat”; if the left part of the brain is working, then it will indicate “Thanks”; if the right part of the brain is working, then it will indicate “Goodbye”; etc. When the brain wave detection device detects the left part of the brain is working, it transmits a preset instruction “Thanks” signal to the processing component, and the processing component makes a sound using the signal through the speaker in the interaction component, so that an idea of the user can be conveyed directly through the speaker to the effect of intelligent interaction.

Based upon the same inventive concept, the embodiments of the disclosure further provides an operating method of the false tooth device according to any one of the embodiments of the disclosure, where the method includes following operations.

The detection component transmits a detected signal to the processing component.

The processing component identifies the signal detected by the detection component as a signal to be interacted with the interaction component, and to transmit the signal to the interaction component.

The interaction component outputs the signal transmitted by the processing component.

In summary, the embodiments of the disclosure provide a false tooth device transmitting a corresponding signal through movements of a mouth, and interacting with a terminal via the signal, the terminal, and a signal interaction system including the false tooth device and the terminal. Where the false tooth device includes a false tooth body, and a processing component, an interaction component, and a detection component arranged in the false tooth body; and in the embodiments of the disclosure, the detection component is arranged in the false tooth body of the false tooth device to detect intuitively in effect a signal conveyed from the movements of the mouth, so that the false tooth device interacts with the terminal via the signal through the signal interaction system, and the terminal converts an idea of a user into a signal, transmits the signal to the false tooth device, and conveys in effect a sound through the false tooth device to thereby facilitate a person vocally disabled.

Those skilled in the art shall appreciate that the embodiments of the disclosure can be embodied as a method, a system or a computer program product. Therefore the disclosure can be embodied in the form of an all-hardware embodiment, an all-software embodiment or an embodiment of software and hardware in combination. Furthermore the disclosure can be embodied in the form of a computer program product embodied in one or more computer useable storage mediums (including but not limited to a disk memory, an optical memory, etc.) in which computer useable program codes are contained.

The disclosure has been described in a flow chart and/or a block diagram of the method, the device (system) and the computer program product according to the embodiments of the disclosure. It shall be appreciated that respective flows and/or blocks in the flow chart and/or the block diagram and combinations of the flows and/or the blocks in the flow chart and/or the block diagram can be embodied in computer program instructions. These computer program instructions can be loaded onto a general-purpose computer, a specific-purpose computer, an embedded processor or a processor of another programmable data processing device to produce a machine so that the instructions executed on the computer or the processor of the other programmable data processing device create means for performing the functions specified in the flow(s) of the flow chart and/or the block(s) of the block diagram.

These computer program instructions can also be stored into a computer readable memory capable of directing the computer or the other programmable data processing device to operate in a specific manner so that the instructions stored in the computer readable memory create an article of manufacture including instruction means which perform the functions specified in the flow(s) of the flow chart and/or the block(s) of the block diagram.

These computer program instructions can also be loaded onto the computer or the other programmable data processing device so that a series of operational steps are performed on the computer or the other programmable data processing device to create a computer implemented process so that the instructions executed on the computer or the other programmable device provide steps for performing the functions specified in the flow(s) of the flow chart and/or the block(s) of the block diagram.

Evidently those skilled in the art can make various modifications and variations to the disclosure without departing from the spirit and scope of the disclosure. Thus the disclosure is also intended to encompass these modifications and variations thereto so long as the modifications and variations come into the scope of the claims appended to the disclosure and their equivalents.

1. A false tooth device, comprising a false tooth body, and a processing component, an interaction component, and a detection component, which are arranged in the false tooth body, wherein:
the detection component is connected with the processing component, and configured to transmit a detected signal to the processing component;
the processing component is connected with the interaction component, and configured to identify a received signal as a signal capable of interacting with the interaction component, and to transmit the received signal to the interaction component; and
the interaction component is configured to output a signal transmitted by the processing component.

2. The false tooth device according to claim 1, wherein the interaction component comprises a radio communication component;
the processing component is configured to identify the received signal as a signal capable of interacting with the radio communication component, and to transmit the received signal to the radio communication component; and
the radio communication component is configured to output the received signal for interaction to a corresponding terminal.

3. The false tooth device according to claim 2, wherein the radio communication component comprises at least one of following components:
a Bluetooth component, a Wireless-Fidelity (Wi-Fi) component, a Zigbee component, a General Packet Radio Service (GPRS) component, and a radio frequency component.

4. The false tooth device according to claim 1, wherein the interaction component comprises a broadcasting component;
the processing component is configured to identify a signal transmitted by the detection component as a voice signal, and to transmit the voice signal to the broadcasting component; and
the broadcasting component is configured to output the voice signal.

5. The false tooth device according to claim 4, wherein the broadcasting component comprises a speaker;
the processing component is configured to identify the signal transmitted by the detection component as the voice signal, and to transmit the voice signal to the speaker; and
the speaker is configured to make a voice corresponding to the voice signal.

6. The false tooth device according to claim 2, wherein the false tooth device further comprises a storage component configured to store a correspondence relationship between a signal detected by the detection component, and the signal capable of interacting with the radio communication component; and
the processing component is configured to identify the signal detected by the detection component as the signal capable of interacting with the radio communication component according to the correspondence relationship.

7. The false tooth device according to claim 1, wherein the false tooth device further comprises a power component configured to provide power.

8. The false tooth device according to claim 1, wherein the detection component comprises at least one of following sensors:
an acceleration sensor, a gyroscope sensor, a pressure sensor, and a Global Positioning System (GPS) sensor.

9. The false tooth device according to claim 2, wherein the interaction component further comprises a speaker;
the radio communication component is further configured to receive a control signal transmitted by the terminal, and to transmit the control signal to the processing component;
the processing component is further configured to identify the control signal as a voice signal, and to transmit the voice signal to the speaker; and
the speaker is configured to make a voice corresponding to the voice signal.

10. A terminal, comprising a communication element configured to perform signal interaction with the false tooth device according to claim 1, and a processing element configured to process a signal received from the false tooth device,
and/or to transmit a signal to the false tooth device through the communication element.

11. The terminal according to claim 10, wherein the terminal comprises at least one of following terminals:
a TV set, a mobile phone, a tablet computer, a watch, and a brain wave detection device.

12. A signal interaction system, comprising the false tooth device claim 1.

13. The signal interaction system according to claim 15, wherein when the interaction component comprises a radio communication component and a speaker, and the terminal comprises a brain wave detection device, the brain wave detection device is configured to transmit an acquired brain wave signal to the processing component in the false tooth device through the radio communication component; the processing component is configured to identify the brain wave signal as a voice signal, and to transmit the voice signal to the speaker; and the speaker is configured to make a voice corresponding to the voice signal.

14. An operating method of the false tooth device according claim 1, the method comprising:
transmitting, by the detection component, the detected signal to the processing component;
identifying, by the processing component, the signal detected by the detection component as the signal capable of interacting with the interaction component, and transmitting the signal to the interaction component; and
outputting, by the interaction component, the signal transmitted by the processing component.

15. The signal interaction system according to claim 12, wherein the signal interaction system further comprises the terminal.
